(19) United States
(12) Patent Application Publication
(10) Pub. No.: US 2009/0259284 A1
(43) Pub. Date: Oct. 15, 2009

(54) Resonating Stent or Stent Element

(75) Inventors: Dwayne S. Yamasaki, Rohnert Park, CA (US); Michael Neidert, Salthill (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/100,574

(22) Filed: Apr. 10, 2008

(51) Int. Cl. A61F 2/82; A61F 2/84; A61N 1/00; A61B 5/06

(52) U.S. Cl. 623/1.11; 623/1.13; 607/116; 600/424

(57) ABSTRACT

A tubular member adapted for endovascular delivery in a human patient comprises a tubular wire framework and the tubular wire framework forms the core of a signal device. The signal device further includes a coil, which is wrapped around the core to form an inductor, and a capacitor coupled to the inductor to form an inductor capacitor circuit. The signal device can be excited and signals acquired therefrom to determine the position and/or orientation of the signal device and the tubular member. The relative positions and/or orientations of (1) the signal device and/or the tubular member and (2) a device having a signal generating element or a signal generating element being advanced toward the signal device, tubular member or framework can be monitored. Relative positions and/or orientations of a plurality of signal devices in a tubular wire framework also can be determined after the framework is deployed in a vessel and compared to predetermined positions and/or orientations.

### FIELD OF THE INVENTION

The invention relates to stents, stent elements, and/or stent-grafts and more particularly to stents or stent elements that have one or more electromagnetic coils integrally formed therein.

### BACKGROUND OF THE INVENTION

Tubular prostheses such as stents, grafts, and stent-grafts (e.g., stents having an inner and/or outer covering comprising graft material and which may be referred to as covered stents) have been widely used in treating abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE) materials) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft spans across and extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. When the prosthesis is a stent-graft, a minimally invasive endovascular approach is preferred by many physicians over traditional open surgery techniques where the diseased vessel is surgically opened, and a graft is sutured into position such that it bypasses an aneurysm. The endovascular approach, which has been used to deliver stents and stent grafts, generally involves cutting through the skin to access a lumen of the vasculature. Alternatively, lumenar or vascular access may be achieved percutaneously via successive dilation at a less traumatic entry point. Once access is achieved, the prosthesis (e.g., a stent-graft) can be routed through the vasculature to the target site. For example, a stent-graft delivery catheter loaded with a stent-graft can be percutaneously introduced into the vasculature (e.g., into a femoral artery) and the stent-graft delivered endovascularly across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter. Upon retraction or removal of the sheath or catheter at the target site, the stent-graft self-expands.

More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to distal end of the inner tube. Once the catheter is positioned for deployment of the stent-graft at the target site, the inner tube is held stationary and the outer tube (sheath) withdrawn so that the stent-graft is gradually exposed and allowed to expand. The inner tube or plunger prevents the stent-graft from moving back as the outer tube or sheath is withdrawn. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Although the endovascular approach is much less invasive, and usually requires less recovery time and involves less risk of complication as compared to open surgery, among the challenges with the approach is positioning the prosthesis and/or locating the prosthesis position.

Generally speaking, physicians often use fluoroscopic imaging techniques to confirm prosthesis position before and during deployment. This approach requires one to administer a radiopaque substance, which generally is referred to as a contrast medium, agent or dye, into the patient so that it reaches the area to be visualized (e.g., the renal arteries). A catheter can be introduced through the femoral artery in the groin of the patient and endovascularly advanced to the vicinity of the renals. The fluoroscopic images of the transient contrast agent in the blood, which can be still images or real-time motion images, allow two-dimensional visualization of the location of the renals. The extensive use of X-rays and cytotoxic contrast that provide known risks from a procedure be carefully balanced with the benefits of the procedure to the patient. While physicians always try to use low dose rates during fluoroscopy, the duration of a procedure may be such that it results in a relatively high absorbed dose to the patient and physician. Patients who cannot tolerate contrast enhanced imaging or physicians who must or wish to reduce radiation exposure need an alternative approach.

Accordingly, there remains a need to develop and/or improve prosthesis positioning and locating apparatus and methods for endoluminal or endovascular applications.

### SUMMARY OF THE INVENTION

Embodiments according to the present invention involve improvements in prosthesis construction to facilitate prosthesis localization (e.g., finding the position of the prosthesis in three-dimensional space), position tracking and/or monitoring.

In one embodiment according to the invention, a tubular member adapted for endovascular delivery in a human patient comprises a tubular wire framework, a coil and a capacitor, a portion of the tubular wire framework forms a core and the coil surrounds the core to form an inductor, the capacitor is coupled to the inductor to form an inductor capacitor circuit. In one example configuration, the inductor capacitor circuit can be an inductor capacitor series circuit.

In another embodiment according to the invention, an endovascular resonating marker assembly comprises a wire stent, a coil, and a capacitor, the coil being wound about a portion of the wire stent to form an inductor and the capacitor being coupled to the portion to form an inductor capacitor circuit.

In another embodiment according to the invention, an endovascular resonating marker assembly comprising a tubular wire stent element, a coil, and a capacitor, the coil being wound about a portion of the wire stent to form an inductor and the capacitor being coupled to the portion to form an inductor capacitor circuit.

In another embodiment according to the invention, a method of endovascularly navigating a device to a prosthesis comprises positioning in a vessel of a patient a prosthesis comprising a tubular wire framework, a coil and a capacitor, where a portion of the tubular wire framework forms a core, the coil surrounds the core to form an inductor and the capacitor is coupled to the inductor to form an inductor capacitor circuit signal element; generating an electromagnetic field in the region of the patient where the prosthesis is positioned; endovascularly advancing a device having an electromagnetic coil secured thereto toward the prosthesis; and monitoring the relative positions of the signal element and the electromagnetic coil based on signals emitted therefrom and indicative of their positions.

In another embodiment according to the invention, a method of evaluating prosthesis deployment comprises endovascularly positioning a prosthesis comprising a tubular wire framework having two resonating markers in a predetermined position; deploying the prosthesis; generating an electromagnetic field in the vicinity of the deployed prosthesis to activate the resonating markers so that they emit signals; determining the position of the markers based on their emitted signals; and evaluating the determined resonating marker positions as compared to the predetermined marker positions.

In another embodiment according to the invention, a method of evaluating prosthesis deployment comprises endovascularly positioning a prosthesis comprising a tubular wire framework having two resonating markers in a predetermined orientation; deploying the prosthesis; generating an electromagnetic field in the vicinity of the deployed prosthesis to activate the resonating markers so that they emit signals; determining the orientation of the markers based on their emitted signals; and evaluating the determined resonating marker orientations as compared to the predetermined marker orientations.

Other features, advantages, and embodiments according to the invention will be apparent to those skilled in the art from the following description and accompanying drawings.

### BRIEF DESCRIPTION OF THE DRAWINGS

FIG. 1 diagrammatically illustrates one embodiment of a stent in accordance with the invention.

FIG. 2 diagrammatically illustrates another embodiment of a stent in accordance with the invention.

FIGS. 3A-3C diagrammatically illustrate components of the stent illustrated in FIG. 1 according to one embodiment, where FIG. 3A illustrates a ferromagnetic core, FIG. 3B illustrates a coil wrapped around the ferromagnetic core FIG. 3B with a capacitor, and FIG. 3C illustrates components of FIG. 3B, including the core, coil, capacitor and portions of stent portions adjacent the core ends, encased.

FIGS. 4A and 4B illustrate one capacitor configuration, where, FIG. 4A illustrates two capacitor elements and FIG. 4B illustrates the capacitor elements of FIG. 4A with insulation therebetween.

FIG. 5 illustrates another capacitor configuration.

FIG. 6 diagrammatically illustrates one configuration of a navigation system in which the stent embodiments described herein can be used.

FIGS. 7A-7C diagrammatically illustrate use of a stent element embodiment according to the invention, where FIG. 7A depicts endovascular prosthesis delivery to a target site; FIG. 7B depicts tracking a catheter into the contralateral gate using resonating stent element assemblies according to the invention and insertion of a guidewire through the catheter after which the catheter is removed to provide a guide for delivery of the contralateral leg of the prosthesis; and FIG. 7C depicts the prosthesis fully deployed with a contralateral leg secured thereto.

FIG. 8A diagrammatically illustrates a known system for energizing and locating leadless electromagnetic markers.

FIG. 8B is a diagrammatical section view of a known leadless electromagnetic marker.

### DETAILED DESCRIPTION

The following description will be made with reference to the drawings where when referring to the various figures, it should be understood that like numerals or characters indicate like elements.

Regarding proximal and distal positions, the proximal end of the prosthesis (e.g., stent-graft) is the end closest to the heart (by way of blood flow) whereas the distal end is the end farthest away from the heart during deployment. In contrast, the distal end of the catheter is usually identified as the end that is farthest from the operator, while the proximal end of the catheter is the end nearest the operator. Therefore, the prosthesis (e.g., stent-graft) and delivery system proximal and distal descriptions may be consistent or opposite to one another depending on prosthesis (e.g., stent-graft) location in relation to the catheter delivery path.

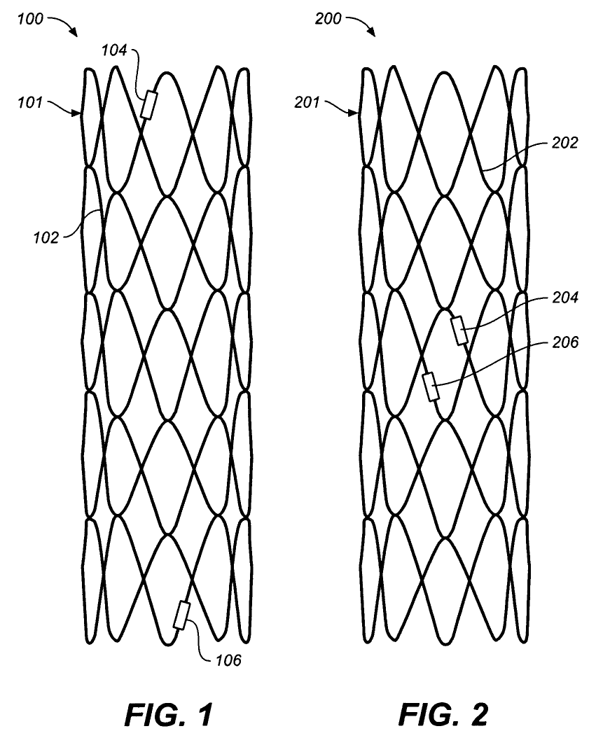

Referring to FIG. 1, one embodiment of an endovascular resonating marker assembly having resonating markers that are suitable for use with a system for generating an excitation field or signal for activating the markers of the endovascular resonating marker assembly and locating the markers of the marker assembly in three-dimensional space is shown and generally designated with reference numeral 100. As will be described in more detail below, an exemplary excitation field or signal generating and marker locating system includes a source generator that generates a selected magnetic excitation field or excitation signal that energizes the markers, which generate measurable marker signals that are measured by a plurality of sensors. The sensors are coupled to a signal processor that uses the measurement of the marker signals from the sensors to calculate the location of each marker in three-dimensional space. An example of a signal generating and marker locating system is described in U.S. Pat. No. 6,822,570 to Dimmer et al. and entitled System for Spatially Adjustable Excitation of Leadless Miniature Marker, the disclosure of which is hereby incorporated herein by reference in its entirety.

Returning to FIG. 1, endovascular resonating marker assembly 100 comprises a stent wire 102, which in the illustrative embodiment forms a plurality of rings that form a tubular stent framework 101 where the rings are secured to each other using traditional means such as variously configured connectors (not shown), direct welding, suture attachment, or attachment to a graft substrate. According to another embodiment, the stent wire can form a single one of the rings shown in FIG. 1 and that single ring can form the framework for an endovascular resonating marker assembly. Further other known stent frameworks can be used as would be apparent to one of ordinary skill in the art.

In the example shown in FIG. 1, endovascular resonating marker assembly 100 further includes resonating markers 104 and 106 integrally formed in the stent framework and positioned in a known orientation relative to one another. In the illustrative example, resonating markers 104 and 106 are positioned close to opposite ends of the stent. In this manner, calculated positions of the markers will approximately correspond to the position of the proximal and distal ends of the stent. More specifically, when acquired navigational data indicative of the position and/or orientation of the markers is sent to a computer or processor for display, the computer can process that information to display a representation of the markers and their relative positions on a display. Alternatively, the relative positions of the markers and the stent and the stent dimensions can be input in the computer so that the computer can process that information to display a representation of the stent.

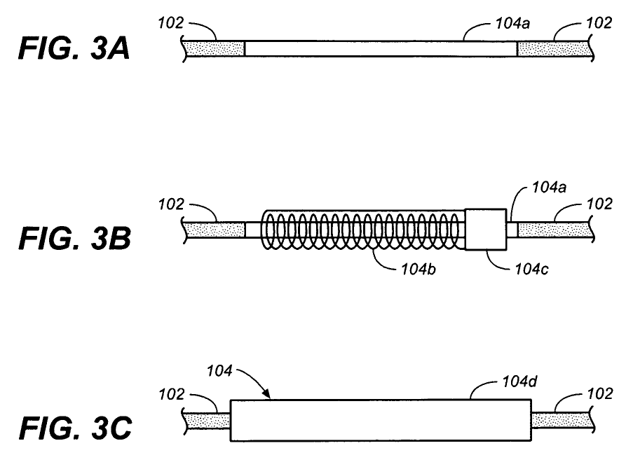

Referring to FIG. 2, another endovascular resonating marker assembly embodiment is shown and generally designated with reference numeral 200. Endovascular resonating marker assembly 200 comprises stent wire 202, which forms framework 201 in the same manner as stent wire 102 forms stent framework 101. According to another embodiment, the stent wire can form a single one of the rings shown in FIG. 2, such as the center marker containing coil, and that single ring can form the framework for an endovascular resonating marker assembly. Further other known stent frameworks can be used as would be apparent to one of ordinary skill in the art.

Endovascular resonating marker assembly 200 further includes resonating markers 204 and 206 integrally formed in the stent framework and positioned in a known or predetermined orientation relative to one another. In the illustrative example, resonating markers 204 and 206 are positioned in a central region of the stent to provide data indicative of where the central region of the state is positioned. More specifically, when acquired navigational data indicative of the position and/or orientation of the markers is sent to a computer or processor for display, the computer can process that information to display a representation of the markers and their relative positions on a display. Alternatively, the relative positions of the markers and the stent and the stent dimensions can be input in the computer so that the computer can process that information to display a representation of the stent.

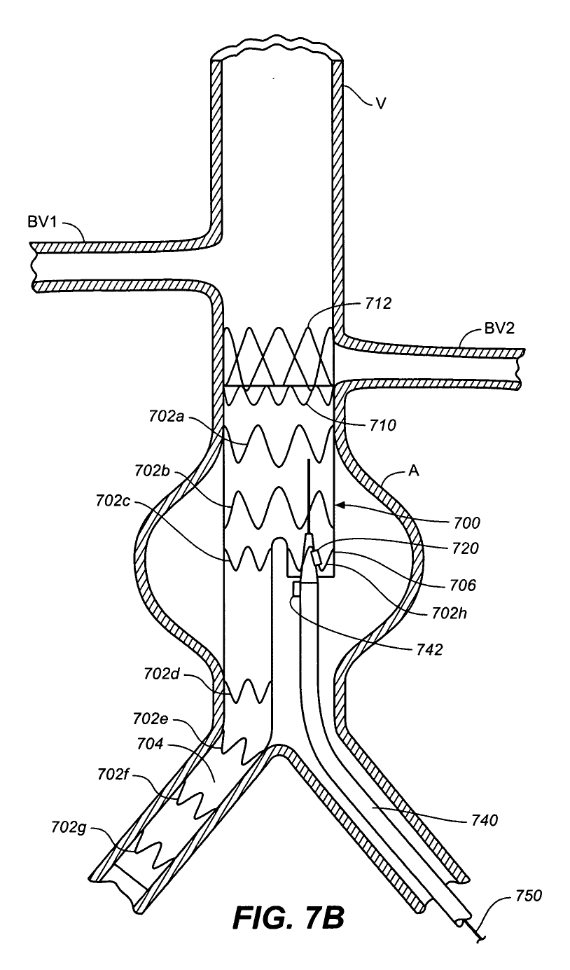

Although two markers are shown in each of the foregoing illustrative embodiments, each assembly can have only one marker. A single marker coil provides position and orientation of the marker coil, which can be used to determine the position and/or orientation of the marker or marker assembly based on the relative positions of the marker, marker coil and stent framework and dimensions of the marker, marker coil and stent framework. Therefore, the single marker coil configuration can provide the vector orientation of the stent framework. However, two or more markers or marker coils can be used. In one example, a two coil configuration can be used to confirm and add reliability to the orientation and position measurements. One can subtract between the marker coils to verify the orientation and use the location of one marker coil as a check on the location of the other marker coil. More specifically, a navigation system such as navigation system 10 as shown in FIG. 6 can be used. Tracking system 10 includes an imaging device 12, tracking system 14, which includes a tracker 20 and tracked elements 22a,b,c . . . n, which would, for example correspond to markers 104 and 106 or markers 204 and 206, and a display coupled to computer or processor 18 to display information regarding the position and/or orientation of one or more tracked elements or the assembly of which they form a part or information regarding the relative positions of the tracked elements. In one example, the tracking system tracks the position of the marker coils, processes the information received from the marker coils when they are activated, and provides five values for each marker coil. Three values correspond to the XYZ coordinates in an XYZ coordinate system that correspond to the position of a marker coil in three-dimensional space. The fourth and fifth values for each marker indicate pitch and yaw of the marker coil, which are angular measurements of the marker coils in three-dimensional space that indicate the direction of the coils. The tracking system can be calibrated to track the center point of the marker coil as is known in the art. When the coil is a symmetrically configured cylinder as shown in the illustrative embodiments, all marker representations provided to the computer for processing and display are of the center point of the marker coil. Returning to the confirmation step, one can check the orientation of the coils and compare that with the known or predetermined orientation of the coils, for example, when resonating marker assembly 100 or 200 is in an expanded state or in the intended configuration when deployed in a patient, but prior to use. This information can be input into the computer as well. Thus, if the tracked coil positions are not approximately parallel as shown in FIG. 2 (see markers 204 and 206), proper deployment may not have occurred. In a further example, the tracked positions of the marker coils measured in an XYZ coordinate system are input into the computer. The computer can be programmed to carry out the subtraction of one tracked position from the other to obtain a vector and evaluate the vector based on known vector analysis techniques to determine if the relative positions of the tracked marker coils are accurate as compared to their known or predetermined relative positions (e.g., when resonating marker assembly 100 or 200 is in an expanded state or the intended configuration when deployed in a patient, but prior to use), which are input into the computer. If the tracked relative positions are significantly different from the known positions, proper deployment may not have occurred. The stent (or stent framework, e.g., stent framework 101 or 201) dimensions, the marker positions, and the marker orientations relative to an aspect of the stent (or stent framework, e.g., stent framework 101 or 201) are input into the computer so that after the computer processes the acquired tracked data, a representation of the marker(s) and/or marker assembly (e.g., marker assembly 100 or 200) post deployment position and orientation can be displayed.

Returning to FIG. 1, similar confirmations to those described above can be made when using at least two markers. Alternatively, one marker can be eliminated and the other positioned at one end of the stent to facilitate monitoring the proximal or distal end of the stent. In a further alternative, the position of the marker relative to the stent framework 101 and the dimensions of stent framework 101 can be input into the computer or processor to display a representation of the stent.

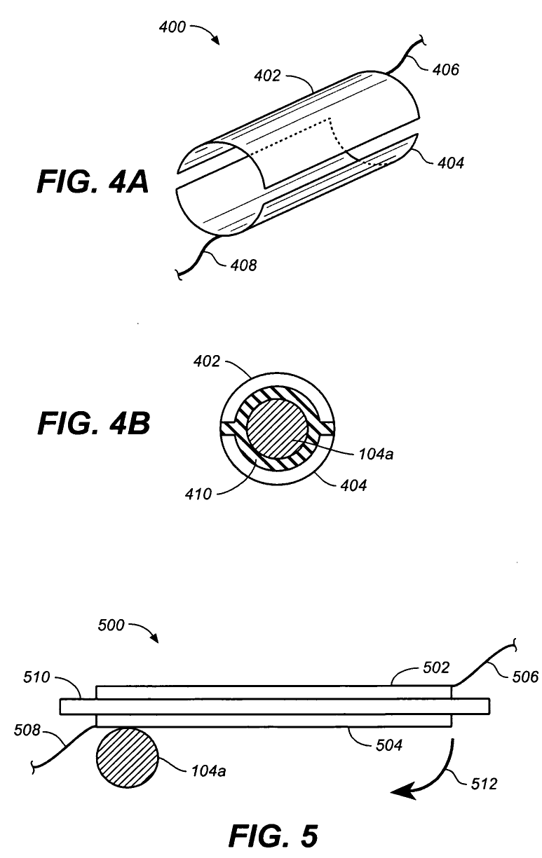

Referring to FIGS. 3A-C, one embodiment of resonating marker 104 is shown. Marker 104 is an inert, activatable device that can be excited to generate a signal at a resonant frequency measurable by a sensor array that is remote from the marker. Resonating member or marker 104 generally comprises a core 104a, coil windings 104b, and capacitor 104c. Coil 104b is wound around core 104a to form an inductor (L). The inductor (L) is connected to capacitor (C), which is designated with reference numeral 104c, so as to form a signal element. Accordingly, the signal element is an inductor (L) capacitor (C) resonant circuit. Although the coil is diagrammatically shown in symbolic fashion, it should be understood that it is tightly wound around core 104a and can be formed from an elongated insulated copper wire (e.g., low resistance, small diameter, insulated wire).

In the illustrative embodiment, the resonating marker 104 also can include a protective encapsulation or casing 104d to protect the signal element when tracked or implanted in a patient's body. Encapsulation or casing 104d seals and/or encapsulates the signal element and can be made of plastic, glass, or other suitable inert material. The signal element can be potted with a silicone type plastic or covered with a thin heat shrink. A PTFE heat shrink is desirable for providing insulation and blood compatibility. The markers can have an axial dimension or length of approximately 2-14 mm and a diameter of approximately 0.5-5 mm.

The core 104a can be the same material as the remainder of stent framework 101 or it can be different material. In the former case, stent framework 101, including core portion 104a, is made from any suitable material that provides the desired magnetic properties. Examples include 400 series stainless steel, a cobalt chrome alloy such as L605, tantalum, a nickel based alloy such as Inconel, shape memory alloys such as nitinol, a platinum-iridium alloy such as MP35N, and a niobium-based alloy C-103. When the core portion and the remainder of stent-framework 101 are made from different materials, the core portion can be a ferromagnetic material. For example, core portion 104a can be ferromagnetic material and the remainder of stent framework 101 can be nitinol. In this case, a portion of stent framework is cut away with two ends exposed. One end of the ferromagnetic core portion is then riveted to one of the stent wire exposed ends and the other end of the ferromagnetic core portion is riveted to the other stent wire exposed end in a manner similar to riveting a fluoroscopic marker to a stent. Further, the core can be provided with diametrically enlarged ferromagnetic end portions, which are not surrounded by coil wire, as described in U.S. Pat. No. 7,135,978 to Gisselberg et al. The end portions or end caps can have an outer diameter approximately the same as the outer diameter of coil 104b.

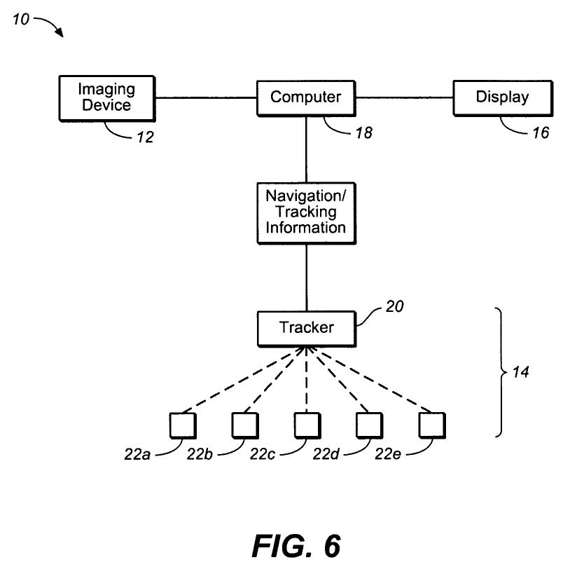

Referring to FIGS. 4A and 4B, one capacitor configuration that can be used is shown and generally designated with reference numeral 400. Capacitor 400 includes two elements or halves 402 and 404, each having a configuration corresponding to a portion of a cylinder. Lead 406 extends from capacitor element 402 and is coupled to one end of the signal element coil (e.g., coil 104b) and lead 408 extends from capacitor element 404 and is coupled to the other end of the signal element coil (e.g., coil 104b). In this embodiment, insulation 410 is fixedly secured to the inner surface of each capacitor half and to the outer surface of the ferromagnetic core (e.g., core 104a) and is configured to separate the capacitor halves. Insulation 410 can comprise any suitable dielectric material.

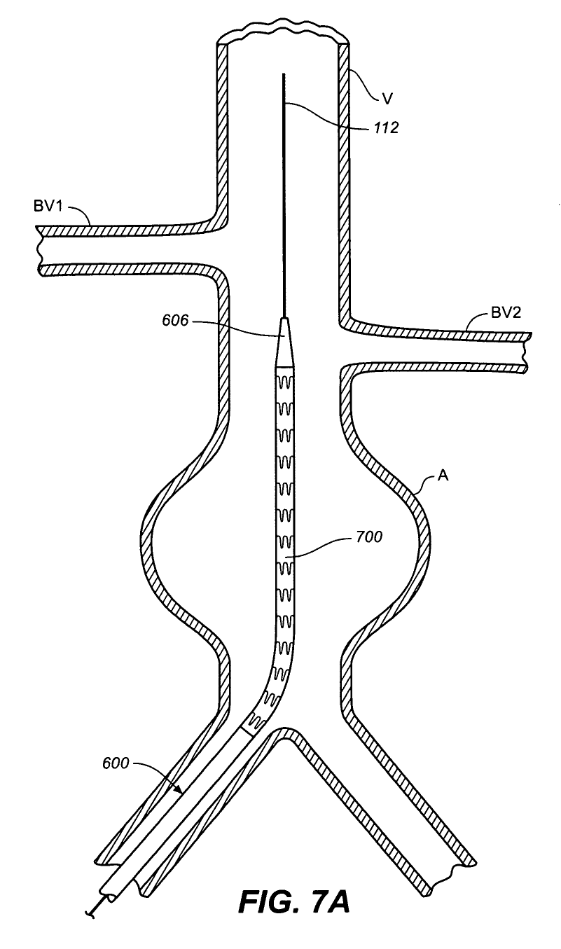

Referring to FIG. 5, another capacitor configuration is shown and generally designated with reference numeral 500. Capacitor 500 comprises first and second flexible capacitor members 502 and 504. Lead 506 extends from capacitor element 502 and is coupled to one end of the signal element coil (e.g., coil 104b) and lead 508 extends from capacitor element 504 and is coupled to one end of the other end of the signal element coil (e.g., coil 104b). In this embodiment, flexible insulation member 510 is fixedly secured to the inner surface of each capacitor member and is configured to separate the capacitor halves. Insulation 510 can be any suitable dielectric material. Capacitor 500 is wrapped around the ferromagnetic core (e.g., core 102a) in the direction of arrow 512. Capacitors formed by rolling up two sheets of conductive material separated by a dielectric material are old and have long been used on electronic circuit boards (computers and otherwise). They are commonly referred to as “tubular capacitors”.

Although particular capacitor constructions have been shown in FIGS. 4A-B and 5, other capacitor constructions can be used as would be apparent to one of ordinary skill in the art. Examples of resonating marker components, including capacitors, that can be incorporated into any of the resonating markers are described in U.S. Patent Application Publication No. 2003/0052785 to Gisselberg et al. and entitled Miniature Resonating Marker Assembly, U.S. Pat. No. 7,135,978 to Gisselberg et al. and entitled Miniature Resonating Marker Assembly, U.S. Pat. No. 6,889,833 to Seiler et al. and entitled Packaged Systems For Implanting Markers In A Patient And Methods For Manufacturing And Using Such Systems, U.S. Pat. No. 6,812,842 to Dimmer and entitled System For Excitation Of Leadless Miniature Marker, U.S. Pat. No. 6,822,570 to Dimmer et al. and entitled System For Spacially Adjustable Excitation Of Leadless Miniature Marker, U.S. Pat. No. 6,838,990 to Dimmer and entitled System For Excitation Of Leadless Miniature Marker, all the disclosures of which are hereby incorporated herein in their entirety by reference thereto.

Use of the resonating marker assemblies illustrated in FIGS. 1 and 2 in a navigation system now will be described with reference to an imaging approach and an iconic representation approach. According to one navigation system embodiment using the imaging approach, representations of tracked elements and/or the assemblies of which they form a part are superimposed on pre-acquired anatomical images in real-time. “Pre-acquired,” as used herein, is not intended to imply any required minimum duration between receipt of the imaging information and displaying the corresponding image. Momentarily storing the corresponding imaging information (e.g., digital signals) in computer memory, while displaying the image (e.g., fluoroscopic image) constitutes pre-acquiring the image. The pre-acquired images can be acquired using fluoroscopic x-ray techniques, CT, MRI, or other known imaging modalities. Representations of surgical or medical devices (e.g., catheters, probes, or prostheses) based on position information acquired from the tracking system can be overlaid on the pre-acquired images of the patient. In this manner, the physician or interventionalist is able to see the location of the surgical device relative to the patient's anatomy.

According to another navigation system embodiment, the navigation system provides, without the use of patient-specific medical images, the position of one or more tracked elements with iconic representations to indicate the positions and/or orientations of the tracked elements, the relative positions and/or orientations of the tracked elements, or the positions and/or orientations of the marker assemblies of which they form a part. In other embodiments, such iconic representations can be displayed with or superimposed on patient-specific medical images.

Referring to FIG. 6 and before describing illustrative examples of use, navigation system 10 will be described in more detail. Imaging device 12, which can correspond to a preoperative or intraoperative imaging device, is coupled to computer 18, which stores and processes the data that the imaging device acquires for display on display 16. Many known imaging systems can be used to acquire preoperative or intraoperative data. One example of an imaging system that can be used to acquire preoperative data is a CT scanner, which generates a three-dimensional (volumetric) image or model from a plurality of cross-sectional two-dimensional images. Another example of a scanner that can be used to acquire preoperative data is a MR scanner, which also can provide a three-dimensional (volumetric) image. Regarding intraoperative data acquisition, navigation using a fluoroscopic two-dimensional system such as the virtual fluoroscopy system described in U.S. Pat. No. 6,470,207, which issued to Simon et al. and is entitled Navigational Guidance Via Computer-Assisted Fluoroscopic Imaging, can be used. Alternatively, a fluoroscopic three-dimensional (volumetric) system such as the O-arm™ imaging system manufactured by Breakaway Imaging Inc. (Littleton, Mass.) can be used as well as other known imaging systems such as other fixed room fluoroscopes that are capable of three-dimensional reconstructions (e.g., Philips Allura with XperCT capability).

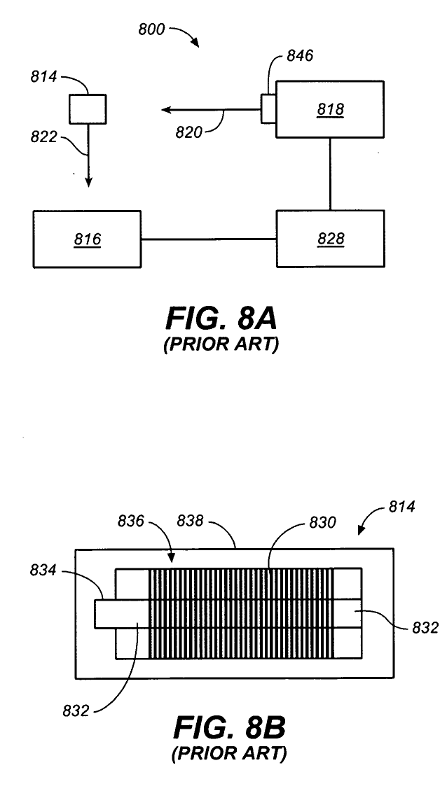

Tracking system 14, which measures positions and/or orientations, and which can for example incorporate known leadless tracking system 800, which is diagramatically shown in FIG. 8A and will be described in more detail below, provides navigational or tracking information to computer 18, which processes that information to display the representations of that information on display 16.

The tracking system typically comprises a tracker 20 and one or more tracked or trackable elements such as 22a, 22b, 22c, 22d, 22e . . . n, which correspond to the markers that form part of resonating marker assembly 100 or 200 and these assemblies can have one or more markers as described above. The tracker provides navigation/tracking information provided to computer 18 so that the position and/or orientation of the marker coils in three-dimensional space can be displayed on display 16 with other marker coils or with a pre-acquired image or superimposed over a pre-acquired image.

When superimposing a tracking system data set over a pre-acquired data set, the data sets are registered. In one example, the preoperative image can be registered via two-dimensional or three-dimensional fluoroscopy. For example, after the preoperative data is acquired, a two-dimensional image is taken intraoperatively and is registered with the preoperative image as is known in the art. Regarding registering two-dimensional and three-dimensional images, see, for example, U.S. Patent Publication No. 2004/021571 to Frank et al. and entitled Method and Apparatus for Performing 2D and 3D Registration, the disclosure of which is hereby incorporated herein by reference in its entirety. In another example, an O-arm™ imaging system manufactured by Breakaway Imaging Inc. (Littleton, Mass.) can be used intraoperatively to take a picture/image of the navigation site to be navigated (see., e.g., U.S. Pat. No. 6,940,941, U.S. to Gregerson et al. and entitled Breakable Gantry Apparatus for Multidimensional X-Ray Based Imaging, U.S. Pat. No. 7,001,045 to Gregerson et al. and entitled Cantilevered Gantry Apparatus for X-Ray Imaging, U.S. Patent Publication No. 2004/0013225 to Gregerson et al. and entitled Systems and Methods for Imaging Large Field-of-View Objects, U.S. Patent Publication No. 2004/0013239 to Gregerson et al. and entitled Systems and Methods for Quasi-Simultaneous Multi-Planar X-Ray Imaging, U.S. Patent Publication No. 2004/0170254 to Gregerson et al. and entitled Gantry Positioning Apparatus for X-Ray Imaging, and U.S. Patent Publication No. 2004/0179643 to Gregerson et al. and entitled Apparatus and Method for Reconstruction of Volumetric Images in a Divergent Scanning Computed Tomography System, the disclosures of which are hereby incorporated by reference in their entirety). Another commercially available system for three-dimensional reconstruction of a volume space is the Innova® 3100 system built on GE's Revolution™ detector technology. A further representative system that performs image registration is described in U.S. Pat. No. 6,470,207 to Simon et al. and entitled Navigational Guidance Via Computer-Assisted Fluoroscopic Imaging, the disclosure of which is hereby incorporated herein by reference in its entirety.

### PROCEDURE EXAMPLE I

A preoperative or intraoperative scan is taken of the target anatomical region where the tracked elements (e.g., resonating markers 104 and 106 or 204 and 206) are to be tracked to obtain a three-dimensional data set of the target anatomical region. The scan can be made using fluoroscopic x-ray techniques, CT, MRI or other known imaging modalities. That information is input into a computer 18, which is programmed to register the acquired data set from the tracker system with the preoperative or intraoperative three-dimensional data set so that one can track the XYZ coordinates of the tracked elements (e.g., resonating markers 104 and 106 or 204 and 206) in the coordinate system of the scanned anatomical structure. Methods for registering such data sets are well known in the art. The tracking system is set to generate electromagnetic energy in a volume of space in which the stent assembly is positioned so that the navigation system can provide the positional data of the markers in an XYZ coordinate system to computer 18, which processes that information to display the position of the stent assembly on the display. The physician or interventionalist introduces a resonating stent assembly (e.g., resonating marker assembly 100 or 200) and delivers it endovascualarly to the target area where a vascular treatment is to be provided, while observing the position of the stent assembly markers superimposed on the three-dimensional image of the anatomical structure on display 16. An optional step of updating the preoperative or intraoperative scan can be included. A commercially available scanner, which acquires an updated three-dimensional data set representative of the target region and simultaneously associates that with a marker, in this example, marker coils 104 and 106 or 204 and 206, can be used. One example is the O-arm™ Imaging System manufactured by Breakaway Imaging Inc. (Littleton, Mass.), which includes navigation software that registers the coordinate systems of the updated three-dimensional data set and the marker. Also see U.S. Pat. No. 6,470,207 to Simon, et al. Another option would be take an ultrasound or fluoroscopic scan to obtain a two-dimensional data set and registering that data set with tracked element data set using established technology such as the FluoroMerge TM™ or FluroNav systems marketed by Medtronic, Inc. (Minneapolis, Minn.).

### PROCEDURE EXAMPLE II

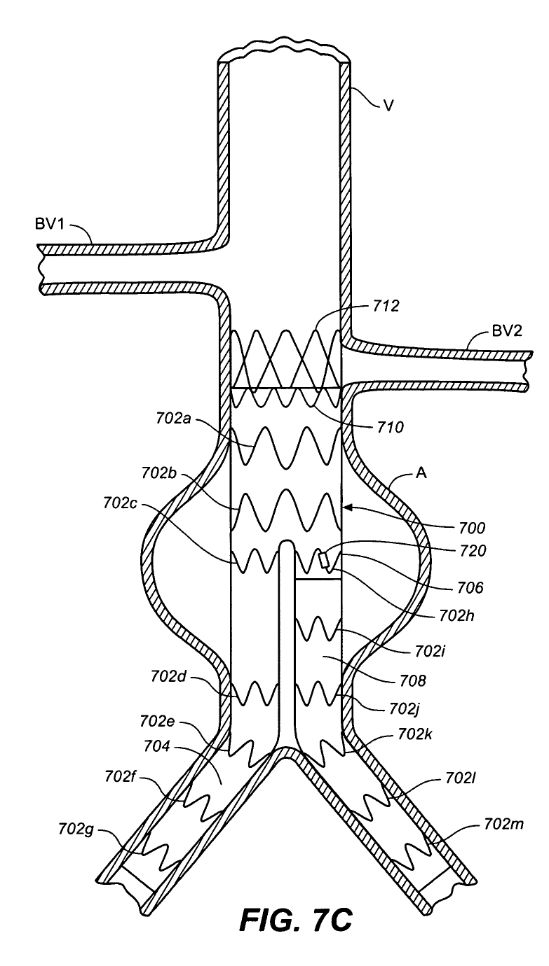

Referring to FIG. 7A, the physician or interventionalist delivers the main body of a bifurcated stent-graft 700 using a stent-graft delivery catheter 600, which includes tapered tip 606, to bypass aneurysm A in vessel V below branch vessels BV1 and BV2, which can correspond to the renal arteries, via the ipsilateral femoral artery. Referring to FIG. 7B, the main body of the stent-graft is shown deployed with resonating marker 720 in the region of its contralateral gate. Resonating marker 720 forms part of undulating wire stent element 702h in the same manner as resonating marker 104 forms part of stent framework 101 and can have the same construction as marker 104. The physician or interventionalist introduces a steerable catheter, having a resonating marker 742 secured to the outer surface of its distal end with glue or other suitable securing means, into the contralateral femoral artery. The steerable catheter can be a conventional steerable catheter having a suitable size for the application, a microcatheter or other suitable catheter. Resonating marker 742 can have the same construction as any of resonating markers 104, 106, 204, and 206 described above. Alternatively, catheter 720 can comprise inner and outer cylindrical extrusions. In this case, a wire coil is wound around the outer surface of the distal end of the inner cylindrical extrusion and the inner extrusion extruded over the inner extrusion to encapsulate the wire coil. The distal end of the inner tube can be made of magnetic material and a tubular split capacitor configuration similar to capacitor 400 described above positioned adjacent to the wire coil with one wire coil lead end connected to one capacitor half portion and the other wire coil lead end connected to the other capacitor half portion before the outer extrusion is extruded over the inner extrusions. The tracking system is set to generate electromagnetic energy in a volume of space in which the bifurcated stent-graft is positioned so that the navigation system can provide the positional data of the marker in an XYZ coordinate system to computer 18, which processes that information to display the position of the bifurcated stent-graft marker 720 on the display. The physician or interventionalist advances the catheter, which has been introduced through the contralateral femoral artery, toward the stent-graft target resonating marker 720 and monitors the relative position of the markers 720 and 742 after the catheter marker 742 enters the excited volumetric space. The tracking system 14 provides data indicative of the positions and/or orientations of the markers in three-dimensional space relative to an XYZ coordinate system and that information is input into the computer, which processes the information to display an iconic representation of the markers on the display. The tracking system provides real time data corresponding to the position of the markers as the catheter is advanced so that the display can provide a real-time representation of the relative position of the markers in three-dimensional space to assist the physician or interventionalist in tracking marker 742 toward marker 720 and cannulating the contralateral gate with catheter 740. After the catheter has passed through contralateral gate, the physician or interventionalist advances a guidewire 750 through the catheter and the contralateral gate, removes the catheter, and tracks a contralateral leg delivery catheter over the guidewire and into the contralateral gate using fluoroscopy. When the contralateral leg is in the desired position, the physician or interventionalist deploys it from its delivery catheter and removes the delivery catheter and guidewire. The full bifurcated stent-graft is shown in FIG. 7C. The stent graft generally includes main body section 704, short leg section 706, and contralateral leg section 708, a plurality of undulating stent elements 702a-m, and an undulating radial support wire 710 all of which are covered with graft material. The stent-graft also can include traditional bare undulating wire 712 extending from the end adjacent branch vessel BV2.

FIGS. 8A and 8B illustrate a system and components for generating an excitation signal for activating a leadless resonating marker assembly and locating the marker in three-dimensional space which can be used in systems for performing methods described herein.

Referring to FIG. 8A, a known leadless electromagnetic system is shown. FIG. 8A is a schematic view of a system 800 for energizing and locating one or more leadless resonating marker assemblies 814 in three-dimensional space relative to a sensor array 816 where one marker assembly 814 is shown in this example. System 800 includes a source generator 818 that generates a selected magnetic excitation field or excitation signal 820 that energizes each marker assembly 814. Each energized marker assembly 814 generates a measurable marker signal 822 that can be sufficiently measured in the presence of both the excitation source signal and environmental noise sources. The marker assemblies 814 can be positioned in or on a selected object in a known orientation relative to each other. The marker signals 822 are measured by a plurality of sensors (not shown) in sensor array 816. The sensors 826 are coupled to a signal processor 828 that utilizes the measurement of the marker signals 822 from the sensors 826 to calculate the location of each marker assembly 814 in three-dimensional space relative to a known frame of reference, such as the sensor array 816.

Source generator 818 is configured to generate the excitation signal 820 so that one or more marker assemblies 814 are sufficiently energized to generate the marker signals 822. The source generator 818 can be switched off after the marker assemblies are energized. Once the source generator 818 is switched off, the excitation signal 820 terminates and is not measurable. Accordingly, sensors 826 in sensor array 816 will receive only marker signals 822 without any interference or magnetic field distortion induced by the excitation signal 820. Termination of the excitation signal 820 occurs before a measurement phase in which marker signals 822 are measured. Such termination of the excitation signal before the measurement phase when the energized marker assemblies 814 are generating the marker signals 822 allows for a sensor array 816 of increased sensitivity that can provide data of a high signal-to-noise ratio to the signal processor 828 for extremely accurate determination of the three-dimensional location of the marker assemblies 814 relative to the sensor array or other frame of reference.

The miniature marker assemblies 814 in the system 800 are inert, activatable assemblies that can be excited to generate a signal at a resonant frequency measurable by the sensor array 816 remote from the target on which they are placed. The miniature marker assemblies 814 have, as one example, a diameter of approximately 2 mm and a length of approximately 5 mm, although other marker assemblies can have different dimensions. An example of such a marker detection systems are described in detail in U.S. Patent Publication No. 2002/0193685 to Mate et al. and entitled Guided Radiation Therapy System, filed Jun. 8, 2001 and published on Dec. 19, 2002, and U.S. Pat. No. 6,822,570 to Dimmer et al., entitled System For Spacially Adjustable Excitation Of Leadless Miniature Marker, all of the disclosures of which are incorporated herein in their entirety by reference thereto.

Referring to FIG. 8B, the illustrated marker assembly 814 includes a coil 830 wound around a ferromagnetic core 832 to form an inductor (L). The inductor (L) is connected to a capacitor 834, so as to form a signal element 836. Accordingly, the signal element 836 is an inductor (L) capacitor (C) resonant circuit. The signal element 836 can be enclosed and sealed in an encapsulation member 838 made of plastic, glass, or other inert material. The illustrated marker assembly 814 is a fully contained and inert unit that can be used, as an example, in medical procedures in which the marker assembly is secured on and/or implanted in a patient's body as described in U.S. Pat. No. 6,822,570 (supra).

The marker assembly 814 is energized, and thus activated, by the magnetic excitation field or excitation signal 820 generated by the source generator 818 such that the marker's signal element 836 generates the measurable marker signal 822. The strength of the measurable marker signal 822 is high relative to environmental background noise at the marker resonant frequency, thereby allowing the marker assembly 614 to be precisely located in three-dimensional space relative to sensor array 816.

The source generator 818 can be adjustable to generate a magnetic field 820 having a waveform that contains energy at selected frequencies that substantially match the resonant frequency of the specifically tuned marker assembly 814. When the marker assembly 814 is excited by the magnetic field 820, the signal element 836 generates the response marker signal 822 containing frequency components centered at the marker's resonant frequency. After the marker assembly 814 is energized for a selected time period, the source generator 818 is switched to the “off” position so the pulsed excitation signal 820 is terminated and provided no measurable interference with the marker signal 822 as received by the sensor array 816.

The marker assembly 814 is constructed to provide an appropriately strong and distinct signal by optimizing marker characteristics and by accurately tuning the marker assembly to a predetermined frequency. Accordingly, multiple uniquely tuned, energized marker assemblies 814 may be reliably and uniquely measured by the sensor array 816. The unique marker assemblies 814 at unique resonant frequencies may be excited and measured simultaneously or during unique time periods. The signal from the tuned miniature marker assembly 814 is significantly above environmental signal noise and sufficiently strong to allow the signal processor 828 (FIG. 8A) to determine the marker assembly's identity, precise location, and orientation in three-dimensional space relative to the sensor array 816 or other selected reference frame.

A system corresponding to system 800 is described in U.S. Pat. No. 6,822,570 to Dimmer et al., entitled System For Spacially Adjustable Excitation Of Leadless Miniature Marker, the entire disclosure of which is hereby incorporated herein in its entirety by reference thereto. According to U.S. Pat. No. 6,822,570, the system can be used in many different applications in which the miniature marker's precise three-dimensional location within an accuracy of approximately 1 mm can be uniquely identified within a relatively large navigational or excitation volume, such as a volume of 12 cm×12 cm×12 cm or greater. One such application is the use of the system to accurately track the position of targets (e.g., tissue) within the human body. In this application, the leadless marker assemblies are implanted at or near the target so the marker assemblies move with the target as a unit and provide positional references of the target relative to a reference frame outside of the body. U.S. Pat. No. 6,822,570 further notes that such a system could also track relative positions of therapeutic devices (i.e., surgical tools, tissue, ablation devices, radiation delivery devices, or other medical devices) relative to the same fixed reference frame by positioning additional leadless marker assemblies on these devices at known locations or by positioning these devices relative to the reference frame. The size of the leadless markers used on therapeutic devices may be increased to allow for greater marker signal levels and a corresponding increase in navigational volume for these devices.

Other examples of leadless markers and/or devices for generating magnetic excitation fields and sensing the target signal are disclosed in U.S. Patent Publication No. 2003/0052785 to Gisselberg et al. and entitled Miniature Resonating Marker Assembly, U.S. Pat. No. 7,135,978 to Gisselberg et al. and entitled Miniature Resonating Marker Assembly, U.S. Pat. No. 6,889,833 to Seiler et al. and entitled Packaged Systems For Implanting Markers In A Patient And Methods For Manufacturing And Using Such Systems, U.S. Pat. No. 6,812,842 to Dimmer and entitled System For Excitation Of Leadless Miniature Marker, U.S. Pat. No. 6,838,990 to Dimmer and entitled System For Excitation Of Leadless Miniature Marker, U.S. Pat. No. 6,977,504 to Wright et al. and entitled Receiver Used In Marker Localization Sensing System Using Coherent Detection, U.S. Pat. No. 7,026,927 to Wright et al. and entitled Receiver Used In Marker Localization Sensing System And Having Dithering In Excitation Pulses all the disclosures of which are hereby incorporated herein in their entirety by reference thereto.

Another example of a suitable leadless marker construction and system is the Calypso® 4D Localization System, which is a target localization platform based on detection of AC electromagnetic markers, called Beacon® transponders, which are implantable devices. These localization systems and markers have been developed by Calypso® Medical Technologies (Seattle, Wash.).

Any feature described in any one embodiment described herein can be combined with any other feature of any of the other embodiments whether preferred or not.

Variations and modifications of the devices and methods disclosed herein will be readily apparent to persons skilled in the art.

What is claimed is:

1. A tubular member adapted for endovascular delivery in a human patient comprising a tubular wire framework, a coil and a capacitor, a portion of said tubular wire framework forms a core and said coil surrounds said core to form an inductor, said capacitor is coupled to said inductor to form an inductor capacitor circuit.

2. The tubular member of claim 1 wherein said wire framework comprises 400 series stainless steel.

3. The tubular member of claim 1 wherein said wire framework comprises a Cobalt Chrome alloy.

4. The tubular member of claim 1 wherein said wire framework comprises tantalum.

5. The tubular member of claim 1 wherein said wire framework comprises a nickel-titanium alloy.

6. The tubular member of claim 1 wherein said wire framework comprises a shape memory alloy.

7. The tubular member of claim 1 wherein said wire framework comprises a platinum-iridium alloy.

8. The tubular member of claim 1 wherein said wire framework comprises a niobium-based alloy.

9. The tubular member of claim 1 wherein said wire framework comprises different materials, and said core comprises ferromagnetic material.

10. The tubular member of claim 1 wherein said tubular member is a stent.

12. The tubular member of claim 1 wherein said tubular member is a stent element.

13. The tubular member of claim 12 further including graft material secured to said stent.

14. The tubular member of claim 1 further including a case that covers said core, coil, and capacitor.

15. An endovascular resonating marker assembly comprising a wire stent, a coil, and a capacitor, said coil being wound about a portion of said wire stent to form an inductor and said capacitor being coupled to said portion to form an inductor capacitor circuit.

16. The endovascular resonating marker assembly of claim 15 wherein said portion comprises magnetic material.

17. The endovascular resonating marker assembly of claim 15 wherein said inductor and capacitor are encapsulated.

18. An endovascular resonating marker assembly comprising a tubular wire stent element, a coil, and a capacitor, said coil being wound about a portion of said wire stent to form an inductor and said capacitor being coupled to said portion to form an inductor capacitor circuit.

19. The endovascular resonating marker assembly of claim 15 wherein said portion comprises magnetic material.

20. The endovascular resonating marker assembly of claim 17 wherein said inductor and capacitor are encapsulated.

21. A method of endovascularly navigating a device to a prosthesis comprising:
positioning in a vessel of a patient a prosthesis comprising a tubular wire framework, a coil and a capacitor, where a portion of the tubular wire framework forms a core, the coil surrounds the core to form an inductor, and the capacitor is coupled to the inductor to form an inductor capacitor circuit signal element;
generating an electromagnetic field in the region of the patient where the prosthesis is positioned;
endovascularly advancing a device having an electromagnetic coil secured thereto toward the prosthesis; and
monitoring the relative positions of the signal element and the electromagnetic coil based on signals emitted therefrom and indicative of their positions.

22. A method of evaluating prosthesis deployment comprising:
endovascularly positioning a prosthesis comprising a tubular wire framework having two resonating markers in a predetermined position;
deploying the prosthesis;
generating an electromagnetic field in the vicinity of the deployed prosthesis to activate the resonating markers so that they emit signals;
determining the position of the markers based on their emitted signals; and
evaluating the determined resonating marker positions as compared to the predetermined marker positions.

23. A method of evaluating prosthesis deployment comprising:
endovascularly positioning a prosthesis comprising a tubular wire framework having two resonating markers in a predetermined orientation;
deploying the prosthesis;
generating an electromagnetic field in the vicinity of the deployed prosthesis to activate the resonating markers so that they emit signals;
determining the orientation of the markers based on their emitted signals; and
evaluating the determined resonating marker orientations as compared to the predetermined marker orientations.

24. The method of claim 23 wherein the predetermined resonating marker orientation corresponds to the resonating markers being approximately parallel.
